(19) United States
(12) Patent Application Publication
(10) Pub. No.: US 2026/0020837 A1
(43) Pub. Date: Jan. 22, 2026

(54) ULTRASOUND IMAGING APPARATUS AND ULTRASOUND IMAGING METHODS

(71) Applicant: SHENZHEN MINDRAY BIO-MEDICAL ELECTRONICS CO., LTD., Shenzhen (CN)

(72) Inventors: Zuci HU, Shenzhen (CN); Wei FAN, Shenzhen (CN); Wenhao ZHANG, Shenzhen (CN)

(73) Assignee: SHENZHEN MINDRAY BIO-MEDICAL ELECTRONICS CO., LTD., Shenzhen (CN)

(21) Appl. No.: 19/270,487

(22) Filed: Jul. 16, 2025

(30) Foreign Application Priority Data
Jul. 16, 2024 (CN) 202410957018.4

(51) Int. Cl. A61B 8/08; A61B 8/00

(52) U.S. Cl. CPC A61B 8/0816; A61B 8/4272

(57) ABSTRACT

Disclosed are an ultrasound imaging apparatus and a ultrasound imaging method. The apparatus comprises: an ultrasound probe comprising a plurality of transducer elements; and a processor configured for: identifying the type and/or depth of an examination site, configuring the ultrasound probe into a first examination mode or a second examination mode based on the identified type and/or depth of the examination site, and generating an ultrasound image of the examination site based on the echo signals received by the ultrasound probe under the first examination mode or the second examination mode. This mode-specific optimization framework allows the first examination mode to suppress near-field grating/side lobe artifacts, while the second examination mode provides improved penetration performance, collectively enhancing image resolution.

### CROSS-REFERENCE TO RELATED APPLICATION

This application claims priority to and the benefit of Chinese Patent Application No. 202410957018.4, filed on Jul. 16, 2024, the disclosure of which is incorporated herein by reference in its entirety.

### TECHNICAL FIELD

The present disclosure relates to the technical field of ultrasound imaging, specifically to ultrasound imaging apparatus and ultrasound imaging methods.

### BACKGROUND

With the rapid development of medical technology, ultrasound imaging technology has become increasingly important and influential. By scanning a specific area with ultrasound beams, performing AD conversion, beam synthesis, and signal processing on the backscattered echo signals, a brightness signal image can be obtained, which is commonly known as a B (Brightness) image. If a certain scanning area is scanned multiple times continuously, and the changes between multiple scans are analyzed, the frequency shift caused by the Doppler effect can be calculated to obtain blood flow signals, which is commonly known as a C (Color) image. In addition to these, there are also imaging methods such as energy and spectrum.

For ultrasound imaging technology, medical staff generally hope that: i. the emitted ultrasound waves have sufficient penetration; ii. the quality of ultrasound images can be continuously improved, such as higher resolution and reduced artifacts caused by grating lobes or side lobes; iii. ultrasound imaging has a sufficient frame rate. How to better obtain ultrasound images that meet the requirements is one of the current problems to be solved or improved.

### SUMMARY

According to a first aspect of the present disclosure, an ultrasound imaging apparatus disclosed in some embodiments may include:
an ultrasound probe, comprising a plurality of transducer elements, and at least configured to emit ultrasound waves to an examination site of an object under examination via at least one of the plurality of transducer elements; and
a processor, configured to:
identify a type and/or depth of the examination site;
configure the ultrasound probe into a first examination mode or a second examination mode based on the identified type and/or depth of the examination site; wherein at least three consecutive transducer elements are present in the plurality of transducer elements, all the at least three consecutive transducer elements are receiving transducer elements when the ultrasound probe is configured into the first examination mode; and when the ultrasound probe is configured into the second examination mode, the first and last transducer elements in the at least three consecutive transducer elements are receiving transducer elements, and at least a non-receiving transducer element is present between the first transducer element and the last transducer element, wherein the receiving transducer element is configured to receive echo signals of the ultrasound waves returned from the examination site, and the non-receiving transducer element is configured not to receive echo signals of the ultrasound waves returned from the examination site; and
generate an ultrasound image of the examination site based on the echo signals received by the ultrasound probe under the first examination mode or the second examination mode; and/or, send echo signals received by the ultrasound probe under the first examination mode or the second examination mode and/or ultrasound data generated based on the echo signals to a terminal device communicatively connected to the ultrasound imaging apparatus, the terminal device being configured to generate an ultrasound image of the examination site based on the received echo signals and/or the ultrasound data.

According to a second aspect of the present disclosure, a cart-based apparatus disclosed in some embodiments may include:
an ultrasound probe, comprising a plurality of transducer elements, and at least configured to emit ultrasound waves to an examination site of an object under examination via at least one of the plurality of transducer elements;
a display device configured to provide a display interface; and
a processor configured to respond to an interactive operation by a user on the display interface to enable the ultrasound probe to enter into a predetermined examination mode, comprising: based on different interactive operations, respectively: enabling the ultrasound probe to enter into a first examination mode, and when the ultrasound probe is operated under the first examination mode, at least three consecutive transducer elements are present in the plurality of transducer elements, all of the at least three consecutive transducer elements are receiving transducer elements; and enabling the ultrasound probe to enter into a second examination mode, and when the ultrasound probe is configured into the second examination mode, the first and last transducer elements among the at least three consecutive transducer elements are receiving transducer elements, and at least a non-receiving transducer element is present between the first transducer element and the last transducer element, wherein the receiving transducer elements are configured to receive echo signals of the ultrasound waves returned from the examination site, and the non-receiving transducer element is configured not to receive echo signals of the ultrasound waves returned from the examination site.

According to a third aspect of the present disclosure, a handheld ultrasound imaging device disclosed in some embodiments may include:
an ultrasound probe, comprising a plurality of transducer elements, and at least configured to emit ultrasound waves to an examination site of an object under examination via at least one of the plurality of transducer elements;
a display device configured to provide a display interface; and
a processor, configured to respond to an interactive operation by a user on the display interface to enable the ultrasound probe to enter into a predetermined examination mode, comprising: based on different interactive operation, respectively: enabling the ultrasound probe to enter into a first examination mode, and when the ultrasound probe is operated under the first examination mode, at least three consecutive transducer elements are present in the plurality of transducer elements, all of the at least three consecutive transducer elements are receiving transducer elements; and enabling the ultrasound probe to enter into a second examination mode, and when the ultrasound probe is configured into the second examination mode, the first and last transducer elements in the at least three consecutive transducer elements are receiving transducer elements, and at least a non-receiving transducer element is present between the first transducer element and the last transducer element, wherein the receiving transducer elements are configured to receive echo signals of the ultrasound waves returned from the examination site, and the non-receiving transducer element is configured not to receive echo signals of the ultrasound waves returned from the examination site;
wherein the display interface is provided by a terminal device communicatively connected to the handheld ultrasound probe.

According to a fourth aspect of the present disclosure, An ultrasound imaging apparatus disclosed in some embodiments may include:
an ultrasound probe comprising a plurality of transducer elements, wherein the ultrasound probe is at least configured to emit ultrasound waves to an examination site of an object under examination by at least one of the plurality of transducer elements; the ultrasound probe at least comprises a first examination mode and a second examination mode; at least three consecutive transducer elements are present in the plurality of transducer elements: when the ultrasound probe is configured into the first examination mode, all the at least three consecutive transducer elements are activated as receiving transducer elements; when the ultrasound probe is configured into the second examination mode, a first transducer element and a last transducer element within the at least three consecutive transducer elements are activated as receiving transducer elements, and at least one non-receiving transducer element is present between the first transducer element and the last transducer element; wherein the receiving transducer elements are configured to receive echo signals of the ultrasound waves returned from the examination site, and the non-receiving transducer element(s) is (are) configured not to receive the echo signals of the ultrasound waves returned from the examination site; and
a processor configured to cause the ultrasound probe to enter into a predetermined examination mode in response to an interactive operation by a user on a display interface, comprising: based on different interactive operations, respectively:
enabling the ultrasound probe to enter into a first examination mode, so as to perform imaging on the examination site under the first examination mode;
enabling the ultrasound probe to enter into a second examination mode, so as to perform imaging on the examination site under the second examination mode; and
wherein the display interface is provided by a terminal device communicatively connected to the ultrasound probe or provided by the ultrasound apparatus.

According to a fifth aspect of the present disclosure, an ultrasound imaging apparatus provided in some embodiments may include:
an ultrasound probe, comprising a plurality of transducer elements, and at least configured to emit ultrasound waves to an examination site of an object under examination via at least one of the plurality of transducer elements; and
a processor, configured to:
identify a type and/or depth of the examination site;
configure the ultrasound probe into a first examination mode or a second examination mode based on the type and/or depth of the examination site, wherein at least three consecutive transducer elements are present in the plurality of transducer elements, when the ultrasound probe is configured into the first examination mode, all of the at least three consecutive transducer elements are receiving transducer elements, and when the ultrasound probe is configured into the second examination mode, at least a non-receiving transducer element is present in the at least three consecutive transducer elements, wherein the receiving transducer elements are configured to receive echo signals of the ultrasound waves returned from the examination site, and the non-receiving transducer element is configured not to receive echo signals of the ultrasound waves returned from the examination site; and generate an ultrasound image of the examination site based on the echo signals received by the ultrasound probe under the first examination mode or the second examination mode; and/or, send echo signals received based on the ultrasound probe under the first examination mode or the second examination mode and/or ultrasound data generated based on the echo signals to a terminal device communicatively connected to the ultrasound imaging apparatus, the terminal device being configured to generate an ultrasound image of the examination site based on the received echo signals and/or the ultrasound data.

According to a sixth aspect of the present disclosure, an ultrasound imaging method provided in some embodiments may include:
identify a type and/or depth of the examination site;
configure an ultrasound probe into a first examination mode or a second examination mode based on the type and/or depth of the examination site, wherein the ultrasound probe comprises a plurality of transducer elements and is at least configured to emit ultrasound waves to an examination site of an object under examination via at least one of the plurality of transducer elements, at least three consecutive transducer elements are present in the plurality of transducer elements, when the ultrasound probe is configured into the first examination mode, all of the at least three consecutive transducer elements are receiving transducer elements, and when the ultrasound probe is configured into the second examination mode, the first and last transducer elements in the at least three consecutive transducer elements are receiving transducer elements, and at least a non-receiving transducer element is present between the first transducer element and the last transducer element, wherein the receiving transducer elements are configured to receive echo signals of the ultrasound waves returned from the examination site, and the non-receiving transducer element is configured not to receive echo signals of the ultrasound waves returned from the examination site; and generate an ultrasound image of the examination site based on the echo signals received by the ultrasound probe under the first examination mode or the second examination mode; and/or, send the echo signals received by the ultrasound probe under the first examination mode or the second examination mode and/or ultrasound data generated based on the echo signals to a terminal device communicatively connected to the ultrasound imaging apparatus, the terminal device being configured to generate an ultrasound image of the examination site based on the received echo signals and/or the ultrasound data.

According to a seventh aspect of the present disclosure, an ultrasound imaging method provided in some embodiments may include:
providing a display interface; and
enabling an ultrasound probe to enter into a predetermined examination mode in response to an interactive operation by a user on the display interface, comprising: based on different interactive operations, respectively: enabling the ultrasound probe to enter into a first examination mode, when the ultrasound probe is operated under the first examination mode, at least three consecutive transducer elements are present among the plurality of transducer elements of the ultrasound probe, and all of the at least three consecutive transducer elements are receiving transducer elements; and enabling the ultrasound probe to enter into a second examination mode, when the ultrasound probe is configured into the second examination mode, the first and last transducer elements in the at least three consecutive transducer elements are receiving transducer elements, and at least a non-receiving transducer element is present between the first transducer element and the last transducer element, wherein the receiving transducer elements are configured to receive echo signals of the ultrasound waves returned from the examination site, and the non-receiving transducer element is configured not to receive echo signals of the ultrasound waves returned from the examination site.

According to a eighth aspect of the present disclosure, an ultrasound imaging method provided in some embodiments may include:
identify a type and/or depth of the examination site;
configure an ultrasound probe into a first examination mode or a second examination mode based on the identified the type and/or depth of the examination site, wherein the ultrasound probe comprises a plurality of transducer elements and is at least configured to emit ultrasound waves to an examination site of an object under examination via at least one of the plurality of transducer elements, at least three consecutive transducer elements are present in the plurality of transducer elements, when the ultrasound probe is configured into the first examination mode, all of the at least three consecutive transducer elements are receiving transducer elements; and when the ultrasound probe is configured into the second examination mode, at least a non-receiving transducer element is present in the at least three consecutive transducer elements, wherein the receiving transducer elements are configured to receive echo signals of the ultrasound waves returned from the examination site, and the non-receiving transducer element is configured not to receive echo signals of the ultrasound waves returned from the examination site; and
generate an ultrasound image of the examination site based on the echo signals receive by the ultrasound probe under the first examination mode or the second examination mode; and/or, send echo signals received by the ultrasound probe under the first examination mode or the second examination mode and/or ultrasound data generated based on the echo signals to a terminal device communicatively connected to the ultrasound imaging apparatus, the terminal device being configured to generate an ultrasound image of the examination site based on the received echo signals and/or the ultrasound data.

In accordance with the ultrasound imaging apparatus and the imaging method thereof mentioned in above embodiments, the ultrasound probe operates in two modes: a first examination mode and a second examination mode. When the ultrasound probe is configured into the first examination mode, all of the at least three consecutive transducer elements function as receiving transducer elements; and when the ultrasound probe is configured into the second examination mode, the first and last transducer elements in the at least three consecutive transducer elements function as receiving transducer elements, with at least a non-receiving transducer element between the first transducer element and the last transducer element. Different examination modes are applied for distinct application scenarios. Under the first examination mode, the receiving transducer elements are adjacent, which avoids grating lobes or side lobes in the near field. Under the second examination mode, at least two receiving transducer elements are spaced apart, expanding the reception aperture to achieve stronger penetration and improved image resolution.

### BRIEF DESCRIPTION OF THE DRAWINGS

FIG. 1 is a schematic diagram of artifacts appearing in an ultrasound image in some embodiments;

FIG. 2 is a schematic diagram of grating lobes or side lobes appearing in an ultrasound image in some embodiments;

FIG. 3 is a schematic diagram of the structure of an ultrasound imaging apparatus in some embodiments;

FIG. 4 is a schematic diagram of the connection between physical channels and transducer elements in some embodiments; and

FIG. 5 is a flowchart of an ultrasound imaging method in some embodiments.

### DETAILED DESCRIPTION

Specific embodiments of the present disclosure will now be described in detail with reference to the accompanying drawings. Similar or related components in different embodiments are labeled with associated reference numerals. The following embodiments include detailed descriptions to facilitate understanding of the present disclosure. However, those skilled in the art will readily recognize that certain features may be omitted under specific circumstances or substituted by other components, materials, or methods. In some instances, certain operations related to the present disclosure are not explicitly described or illustrated herein. This intentional exclusion is intentional to avoid obscuring the core technical solutions of the present disclosure. For those skilled in the art, a complete understanding of these operations can be attained through the descriptions provided in this specification and general technical knowledge in the art.

Additionally, the features, operations, or characteristics described in the specification may be combined in any suitable manner to form various embodiments. Similarly, steps or actions in the method descriptions may be reordered or modified in ways that would be obvious to those skilled in the art. Therefore, the sequences presented in the specification and drawings are intended solely to clarify the description of specific embodiments and do not imply mandatory orderings, unless explicitly stated that a particular sequence is required.

The numerical designations assigned to components in this specification, such as ‘first,’ ‘second,’ or similar ordinal terms, serve solely to distinguish described objects and carry no inherent sequential or technical implications. Furthermore, the terms ‘connected’ and ‘coupled’ as used herein encompass both direct and indirect connection (coupling), unless explicitly stated otherwise.

In some embodiments of the present disclosure, the transducer elements are classified into receiving transducer elements and non-receiving transducer element based on whether they receive the echo signals of the ultrasound waves from the examination site. Specifically, the receiving transducer elements are configured to receive echo signals of the ultrasound waves returned from the examination site, while the non-receiving transducer elements are configured not to receive echo signals of the ultrasound waves returned from the examination site. It should be understood that the concepts of receiving and non-receiving transducer elements are relative. For example, when an ultrasound probe with four transducer elements emits ultrasound waves twice, a first transducer element and a third transducer element receive echo signals of the ultrasound waves during the first ultrasound transmission, making them receiving transducer elements while a second transducer element and a fourth transducer element become non-receiving transducer elements; and the second transducer element and the fourth transducer element receive echo signals of the ultrasound waves during the second ultrasound transmission, making the second transducer element and the fourth transducer element receiving transducer elements while the first transducer element and the third transducer element become non-receiving transducer elements.

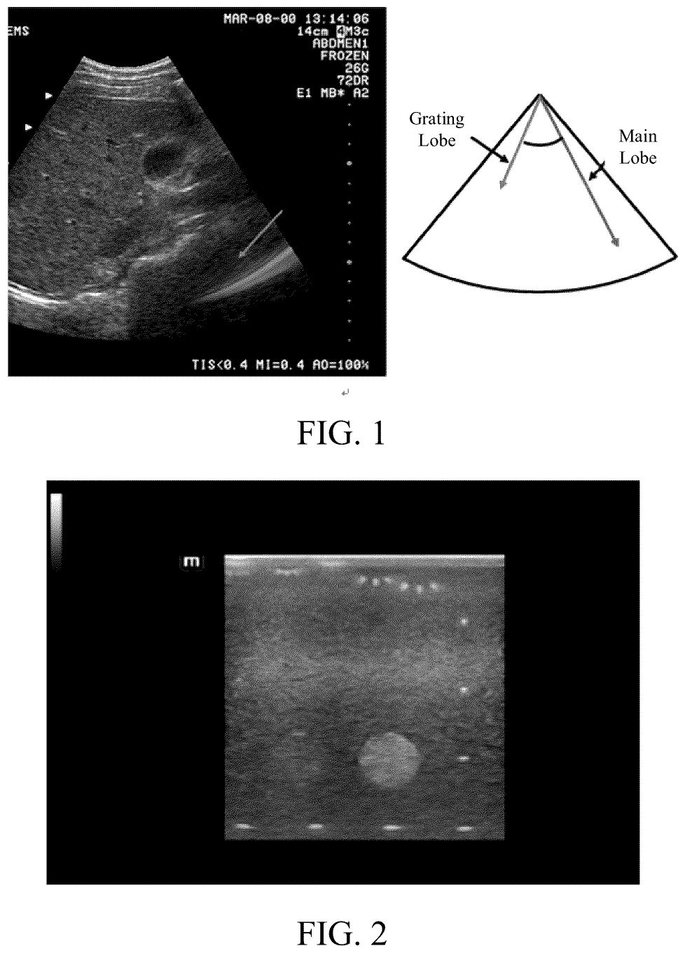

The inventors have observed through clinical practice that when receiving transducer elements are spaced apart, the ultrasound probe can exhibit stronger penetration capability and better image quality in the far-field. However, since the receiving transducer elements are arranged with intervals, the receiving interval is doubled. Given that current ultrasound probes operate with high transmission frequencies, grating lobes or side lobes may occur in the near-field (the ultrasound beam emitted by the ultrasound probe has a main lobe and grating lobes or side lobes, which contain certain ultrasonic energy distributed at angles deviating from the main lobe direction. Since the ultrasound probe cannot distinguish echo signals from the main lobe, grating lobes, or side lobes, the ultrasound imaging apparatus displays echo signals from the main lobe, grating lobes, or side lobes on the same frame image, resulting in echo overlap and artifacts, as shown in FIG. 1). Under the current configuration of spaced receiving transducer elements, significant grating lobes or side lobes appear in the near-field, degrading image quality, as shown in FIG. 2.

The core inventive concept of the present application lies in employing distinct examination modes for different application scenarios. Specifically, the first examination mode avoids grating lobes or side lobes in the near-field, while the second examination mode enhances penetration capability and improves image resolution.

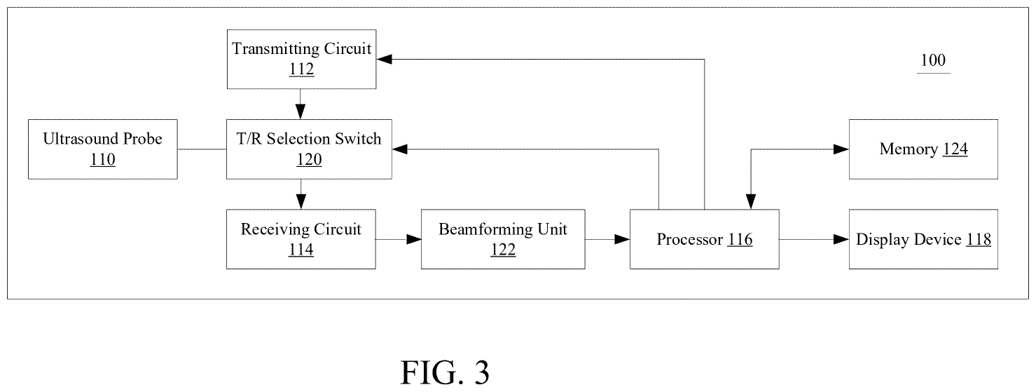

As shown in FIG. 3, the ultrasound imaging apparatus 100 comprises an ultrasound probe 110, a transmitting circuit 112, a receiving circuit 114, a processor 116, and a display device 118. The ultrasound imaging apparatus may further include a transmit/receive (T/R) selection switch 120 and a beamforming unit 122. The transmitting circuit 112 and receiving circuit 114 may be connected to the ultrasound probe 110 via the T/R selection switch 120.

The ultrasound probe 110 comprises multiple transducer elements. These transducer elements may be arranged in a single row to form a linear array, configured as a two-dimensional matrix to create a matrix array, or organized to form a convex array. The transducer elements are configured to emit ultrasound waves based on excitation electrical signals and convert received ultrasound waves into electrical signals. Each transducer element thereby facilitates mutual conversion between electrical pulse signals and ultrasound waves, enabling both transmission of ultrasound waves into tissue within a target region of an object under examination and reception of ultrasound echo waves reflected from said tissue. During ultrasound imaging, transmission sequences and reception sequences may be employed to control which transducer elements are designated for transmitting ultrasound waves and which are allocated for receiving ultrasound waves, alternatively to coordinate the time-division multiplexing of transducer elements between transmitting ultrasound waves and receiving corresponding echo signals. The transducer elements participating in ultrasound wave transmission may be simultaneously excited by electrical signals to achieve concurrent ultrasound emission; alternatively, the transducer elements engaged in ultrasound beam transmission may be sequentially activated by electrical signals with predetermined time intervals, thereby generating sustained ultrasound emissions separated by specified temporal gaps.

During an ultrasound imaging process, the transmitting circuit 112 transmits delayed and focused transmitting pulses to the ultrasound probe 110 via the transmit/receive (T/R) selection switch 120. The ultrasound probe 110, excited by the transmitting pulses, emits ultrasound beams to an examination site of an object under examination. After a predetermined delay, the ultrasound probe 110 receives ultrasound echoes reflected from the examination site carrying tissue information, and converts the ultrasound echoes back into electrical signals. The object under examination may be a human, or alternatively, the object under examination may be an animal such as a cat, dog, rabbit, or the like. The receiving circuit 114 receives the electrical signals converted by the ultrasound probe 110 to obtain ultrasound echo signals, and transmits these ultrasound echo signals to the beamforming unit 122. The beamforming unit 122 processes the ultrasound echo signals through focusing delay, weighting, and channel summation operations, then delivers the processed signals to the processor 116. The processor 116 performs signal detection, signal enhancement, data conversion, and logarithmic compression on the ultrasound echo signals to generate an ultrasound image. The ultrasound image produced by the processor 116 may be displayed on the display device 118 or stored in a memory 124.

Optionally, the processor 116 may be implemented as software, hardware, firmware, or any combination thereof, and may utilize one or more application specific integrated circuits (ASICs), one or more general purpose integrated circuits, one or more microprocessors, one or more programmable logic devices (PLDs), any combination of the aforementioned circuits and/or devices, or other suitable circuits or devices. Furthermore, the processor 116 may control other components in the ultrasound imaging apparatus 100 to execute corresponding steps of the methods described in various embodiments of this specification.

The display device 118, operatively coupled to the processor 116, may be a touch display screen, a liquid crystal display (LCD), or the like; alternatively, the display device 118 may be a standalone display external to the ultrasound imaging apparatus 100, such as an LCD monitor, television set, or other independent display; or alternatively, the display device 118 may be a display screen of an electronic device such as a smartphone, tablet computer, or similar device. The display device 118 may include one or more display units. For example, the display device 118 may comprise a main screen and a touch screen, wherein the main screen is primarily configured for displaying ultrasound images and the touch screen is primarily used for human-machine interaction.

The display device 118 may be configured to display ultrasound images generated by the processor 116. Furthermore, while displaying the ultrasound images, the display device 118 may simultaneously provide a graphical interface containing one or more control elements to enable user interaction. The graphical interface is configured to receive operational commands through a human-machine interaction device for controlling said control elements, thereby executing corresponding control operations. By way of example, the graphical interface may display an icon operable via the human-machine interaction device to perform specific functions, including but not limited to rendering a region-of-interest (ROI) box overlay on the ultrasound images.

Optionally, the ultrasound imaging apparatus 100 may further comprise additional human-machine interaction devices beyond the display device 118, operatively coupled to the processor 116. For example, the processor 116 may be connected with the human-machine interaction devices through an external input/output (I/O) port. The external I/O port may be implemented as a wireless communication module, a wired communication module, or a combination thereof. The external I/O port may be configured to operate based on universal serial bus (USB) standards, controller area network (CAN) bus protocol, and/or wired network protocols.

The human-machine interaction devices may include an input device configured to detect user input information. Said input information may include, but is not limited to: control instructions for ultrasound transmission/reception timing sequences; operational input commands for rendering points, lines, or bounding boxes on ultrasound images; and other types of instructions. The input device may include one or more of the following: a keyboard, a mouse, a scroll wheel, a trackball, a mobile input device (e.g., a touch-enabled mobile device, a smartphone, and the like), a multifunctional knob, or combinations thereof. The human-machine interaction devices may further include output devices such as printers.

The ultrasound imaging apparatus 100 may further comprise a memory 124 configured to: store instructions executable by the processor; store received ultrasound echoes; and store ultrasound images. The memory may include, but is not limited to, a flash memory card, solid-state memory, hard disk drive, or similar storage media. The memory may be implemented as volatile memory and/or non-volatile memory, and may be configured as removable memory and/or non-removable memory.

It should be understood that the components included in the ultrasound imaging apparatus 100 illustrated in FIG. 3 are schematic representations. The apparatus may include more or fewer components than those depicted. For instance, the ultrasound imaging apparatus 100 may be configured as a cart-based ultrasound system, a portable ultrasound scanner, a handheld ultrasound device, or similar configurations, and is not limited to the exemplified configurations under the present application.

In some embodiments, the processor 116 determines at least one of a type and a depth of the examination site, and configures the ultrasound probe 110 into a first examination mode or a second examination mode based on the determined type and/or depth of the examination site. The depth in the present embodiment may be characterized by either: (a) a distance between the examination site and a body surface, or (b) a distance between the examination site and the ultrasound probe 110.

By way of example, the processor 116 first acquires an initial image of the examination site. For instance, said initial image is generated based on echo signals received after the ultrasound probe 110 transmits ultrasound waves toward the examination site. The processor 116 then analyze the initial image of the examination site to determine at least one of the type and depth of the examination site.

In some embodiments, based on the acquired initial image of the examination site, at least one of the type and the depth of the examination site is automatically identified using image recognition algorithms. Such image recognition algorithms are mature prior art techniques that are not repeated herein.

In other embodiments, the display device 118 may provide a display interface configured to: (i) present type identifiers of the examination site after empirical confirmation of the image type via the interface, and (ii) identify the type of the examination site based on user selection of the type identifiers. That is, the type of the examination site may be determined through user interaction with the display interface.

In some embodiments, the display interface may further display a depth input field and/or depth selection options. The depth of the examination site is identified based on user interaction with the depth input field and/or depth selection options. As an example: the depth input field may be a text entry box or input window where a user inputs numerical values to specify the depth of the examination site; and the depth selection options may be a pre-configured drop-down menu containing selectable preset depths such as 5 cm, 10 cm, 15 cm, etc., wherein the user selects one of them to input the depth of the examination site.

The following example illustrates how the examination mode is determined based on the type of examination site.

When the identified type of examination site is thyroid, carotid artery, or blood vessels, the ultrasound probe 110 is set to the first examination mode; and when the identified type is abdomen or heart, the ultrasound probe 110 is set to the second examination mode. The abdomen specifically refers to small organs within the abdominal region, including intestines, kidneys, stomach, and glands. The types of examination sites listed above are provided merely as examples. As demonstrated in these examples, when the examination site pertains to or includes areas relatively close to the body surface, the first examination mode is employed. Conversely, when the examination site pertains to or includes areas located relatively far from the body surface, the second examination mode is utilized.

The following is an example illustrating how to determine the examination mode based on depth.

When the identified depth of the examination site is within a first depth range, the ultrasound probe 110 is set to the first examination mode; and when the identified depth of the examination site is within a second depth range, the ultrasound probe 110 is set to the second examination mode; wherein a lower limit of the second depth range is greater than an upper limit of the first depth range. For example, the upper limit of the first depth range is less than or equal to 5 centimeters, and the lower limit of the second depth range is greater than or equal to 15 centimeters. As another example, the upper limit of the first depth range is less than 10 centimeters, and the lower limit of the second depth range is greater than or equal to 10 centimeters.

In some embodiments, in response to an interactive operation by a user on the display interface, the ultrasound probe 110 is caused to enter into the first examination mode or the second examination mode. For example, the display interface may display an identifier corresponding to the first examination mode or an identifier corresponding to the second examination mode, wherein the ultrasound probe 110 is switched to the examination mode associated with the selected identifier based on the user's selection.

In some embodiments, the system may bypass performing image recognition on the initial image, such as in cases where the user definitively knows the site to be examined. The user may directly manually select a corresponding examination mode identifier on the display interface or perform an operation through a depth input field/selection option, thereby causing the system to enter into the examination mode associated with the selected identifier or the manually specified depth value.

When the ultrasound probe 110 is set to the first examination mode, all transducer elements within at least three consecutive transducer elements are configured as receiving transducer elements. For example, the ultrasound probe 110 comprises 256 transducer elements, and all the 256 transducer elements are activated as receiving transducer elements during each ultrasound transmission; alternatively, the 128 transducer elements are divided into four groups with 64 transducer elements per group, during each ultrasound transmission, one group of 64 transducer elements is functioned as receiving transducer elements, while the remaining three groups are configured as non-receiving transducer elements, and the four groups cycle sequentially as receiving transducer elements during successive transmissions.

In the first examination mode, the continuity of the receiving transducer elements suppresses the occurrence of grating lobes/side lobes in the near-field region, thereby minimizing the generation of artifacts in subsequently generated ultrasound images. This configuration ensures enhanced imaging quality for superficial anatomical sites (e.g., those close to the body surface).

In some embodiments, when the ultrasound probe 110 is configured into the second examination mode, both the first transducer element and the last transducer element among at least three consecutive elements are receiving transducer elements, with at least one non-receiving transducer element presented between the first transducer element and the last transducer element. For example, the ultrasound probe 110 comprises four elements where the first transducer element and the fourth transducer element are receiving transducer elements while the second transducer element and the third transducer element are non-receiving transducer elements. As another example, the first, second, and fourth transducer elements are receiving transducer elements while the third transducer element is a non-receiving transducer element. In some embodiments, any two receiving transducer elements are non-adjacent, for example, one or more non-receiving transducer elements are disposed between any two receiving transducer elements. Furthermore, the receiving transducer elements may be uniformly spaced apart. For instance, one non-receiving transducer element may be positioned between every two receiving transducer elements, or two non-receiving transducer elements may be positioned between every two receiving transducer elements.

In some embodiments, when the ultrasound probe 110 is configured into the second examination mode, at least one non-receiving transducer element exists among at least three consecutive elements. In such embodiments, one of the first and last elements may not be a receiving transducer element, or both may not be receiving transducer elements. For example, the ultrasound probe 110 comprises eight elements, where the odd-numbered elements are receiving transducer elements and the even-numbered transducer elements are non-receiving transducer elements. Similarly, in scenarios where at least one non-receiving transducer element exists among at least three consecutive transducer elements, in some embodiments, any two receiving transducer elements are non-adjacent, with one or more non-receiving transducer elements disposed between them. Further, the receiving transducer elements may be uniformly spaced apart. For instance, one non-receiving transducer element may be positioned between every two receiving transducer elements, or two non-receiving transducer elements may be positioned between every two receiving transducer elements.

It should be noted that when the ultrasound probe 110 is configured into the second examination mode, the designation of receiving transducer elements and non-receiving transducer elements is defined relative to the reception of the same ultrasound wave emission. For example, for an ultrasound probe 110 comprising four elements: during the first reception of echo signals of the ultrasound waves, the first and fourth transducer elements are receiving transducer elements while the second and third are non-receiving transducer elements; and during the second reception of echo signals of the ultrasound waves, the first and third transducer elements are receiving transducer elements while the second and fourth are non-receiving transducer elements. In this scenario, although the third and fourth transducer elements may both act as receiving transducer elements under the second examination mode, they do not constitute consecutive receiving transducer elements within the context of the second examination mode described in this embodiment.

In comparison to the first examination mode, the second examination mode described above can increase the receive aperture while maintaining the same number of receiving transducer elements. For example, with 32 receiving transducer elements: under the first examination mode, the receive aperture spans 32 elements, whereas under the second examination mode, under the configuration of uniformly spacing one non-receiving transducer element between receiving transducer elements (e.g., odd-numbered elements as receiving transducer elements and even-numbered elements as non-receiving transducer elements), the 32 receiving transducer elements would include the 1st, 3rd, 5th, . . . , 63rd elements, while the 2nd, 4th, 6th, . . . , 64th elements are non-receiving transducer elements. This results in a receive aperture spanning 64 elements. Since the receiving transducer elements are interleaved, the inter-element spacing is doubled, thereby enhancing penetration performance. That is, this configuration achieves clearer imaging in far-field regions and provides superior visualization for examination sites deeper beneath the body surface.

As can be seen from the foregoing description, the ultrasound probe 110 in the above embodiments can be adapted for different imaging application scenarios: when performing superficial examinations focusing on near-field imaging, the first examination mode is employed; whereas for applications such as abdominal examinations where greater emphasis is placed on far-field imaging, the second examination mode is adopted to enhance penetration performance. In summary, by utilizing different examination modes for distinct application scenarios, this configuration improves image resolution while avoiding grating lobe artifacts that might otherwise occur in near-field observations.

In some embodiments, the connection scheme between the physical channels and transducer elements is specially designed to enable switching between the first examination mode and the second examination mode as described in some embodiments of the present application. Specifically:

The ultrasound probe 110 further comprises a plurality of physical channels, where the number of transducer elements is M and the number of physical channels is N. Here, N is an even integer greater than or equal to 2, and Mis K times of N, where K is a positive integer greater than or equal to 2. The ((k−1)*N+n)-th transducer element is connectable to the n-th physical channel, where k=1, 2, . . . , K and n=1, 2, . . . , N. The physical channels are configured to excite the connected transducer elements to emit ultrasound waves or to receive echo signals of the ultrasound waves through the connected transducer elements. For example, N may take values such as 8, 16, 32, 64, 128, 192, or 256, while M may take values such as 64, 80, 96, 128, 192, 256, 336, etc.

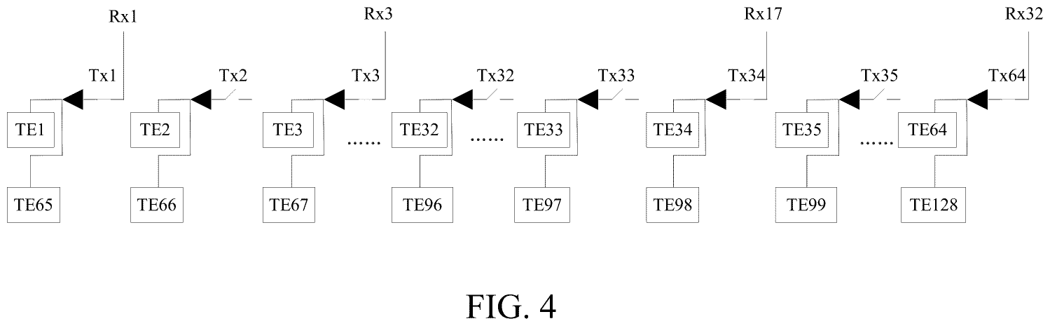

Exemplarily, N is 64, K is 2, and M is 256. FIG. 4 illustrates the connection manner of the 64 physical channels to the first 128 transducer elements (TEs), while the connection manner of the 64 physical channels to the latter 128 transducer elements is similar. Specifically: the first physical channel connects to the 1st transducer element (TE1) and the 65th transducer element (TE65); the second physical channel connects to the 2nd transducer element (TE2) and the 66th transducer element (TE66); . . . the 32nd physical channel connects to the 32nd transducer element (TE32) and the 96th transducer element (TE96); the 35th physical channel connects to the 35th transducer element (TE35) and the 99th transducer element (TE99); . . . the 64th physical channel connects to the 64th transducer element (TE64) and the 128th transducer element (TE128). In the following examples, the connection configuration between physical channels and transducer elements shown in FIG. 4 is adopted. In FIG. 4: the receive selection switch on the first physical channel is closed, while the receive selection switch on the second physical channel is open; and so on. The receive selection switch on the first physical channel is denoted as TX1, the receive selection switch on the second physical channel is denoted as TX2, and so on. RX represents the transmit selection switch, for example, RX1 represents the first transmit selection switch.

When the ultrasound probe 110 is set to the first examination mode, the first to the N-th, the (N+1)-th to the (2*N)-th . . . or the ((K−1)*N+1)-th to the (K*N)-th transducer elements are all receiving transducer elements.

For example, under the first examination mode, the receive selection switches on the first physical channel to the 64th physical channel are controlled to be closed, and then the first physical channel is mapped to the first transducer element, the second physical channel is mapped to the second transducer element, and so on, so that the first transducer element to the 64th transducer element are receiving transducer elements. The above-mentioned “map” can be achieved through software in this field and will not be repeated here. It may be understood that the first physical channel may also be mapped to the 65th transducer element, the second physical channel to the 66th transducer element, and so on, so that the 65th transducer element to the 128th transducer element are receiving transducer elements. It can be seen that the receiving aperture formed above includes 64 consecutive transducer elements.

When the ultrasound probe 110 is set to the second examination mode, the 1st, 3rd, . . . , to (N−1)-th, and the (N+2)-th, (N+4)-th, . . . , to (2N)-th transducer elements are all receiving transducer elements, and the 2nd, 4th, . . . , to N-th, and the (N+1)-th, (N+3)-th, . . . , (2N−1)-th transducer elements are all non-receiving transducer elements; or, the (N+1)-th, (N+3)-th, . . . , to the (2N−1)-th, and the (2N+2)-th, (2N+4)-th, . . . , to the (3N)-th transducer elements are all receiving transducer elements, and the (N+2)-th, (N+4)-th, . . . , to the (2N)-th, and the (2N+1)-th, (2N+3)-th, . . . , to the (3N−1)-th transducer elements are all non-receiving transducer elements; . . . or, the ((K−2)*N+1)-th, ((K−2)*N+3)-th, . . . to the ((K−1)*N−1)-th and the ((K−1)*N+2)-th, ((K−1)*N+4)-th, . . . to the (K*N)-th transducer elements are all receiving transducer elements, and the ((K−2)*N+2)-th, ((K−2)*N+4)-th, . . . to the ((K−1)*N)-th and the ((K−1)*N+1)-th, ((K−1)*N+3)-th, . . . to the (K*N−1)-th transducer elements are all non-receiving transducer elements.

Continuing with the connection method shown in FIG. 4 as an example, under the second examination mode, the receive selection switches on the first physical channel to the 64th physical channel are closed. Then, the first physical channel is mapped to the first transducer element, the second physical channel is mapped to the 66th transducer element, the third physical channel is mapped to the third transducer element, the fourth physical channel is mapped to the 68th transducer element, . . . , the 35th physical channel is mapped to the 35th transducer element, . . . , and the 64th physical channel is mapped to the 128th transducer element. It can be seen that the receiving aperture formed in this way consists of 128 transducer elements, with the receiving transducer elements distributed at intervals. This special receiving method can double the receiving aperture and improve the imaging lateral resolution.

Whether under the first examination mode or the second examination mode, an ultrasound image of the examination site can ultimately be generated. Specifically:

In some embodiments, the processor 116 generates an ultrasound image of the examination site based on the echo signals received by the ultrasound probe 110 under the first examination mode or the second examination mode. The ultrasound image is displayed on the display device 118 of the ultrasound imaging apparatus itself.

In some other embodiments, the processor 116 itself does not generate the ultrasound image, but instead sends the echo signals received by the ultrasound probe 110 in the first or second examination mode and/or the ultrasound data generated based on the echo signals to a terminal device that is in communication with the ultrasound imaging apparatus. The terminal device includes, but is not limited to, a smartphone or a tablet computer. The terminal device generates the ultrasound image of the examination site based on the received echo signals and/or the ultrasound data. For example, the terminal device includes a terminal processor that generates the ultrasound image of the examination site based on the received echo signals and/or the ultrasound data. The ultrasound image generated by the terminal device can be sent back to the ultrasound imaging apparatus for display, or the terminal device can provide its own display interface for displaying the ultrasound image. In some embodiments, the ultrasound imaging apparatus is a handheld ultrasound imaging device. Compared with general ultrasound devices, the handheld ultrasound imaging device has a built-in communication module and a smaller overall volume, and can be in communication with the terminal device, thereby enabling the generated ultrasound image to be sent to the terminal device, or the echo signals received by the ultrasound probe 110 and/or the ultrasound data generated based on the echo signals to be sent to the terminal device. In the scenario where the ultrasound apparatus is a handheld ultrasound device, for the processing of the ultrasound image, it can be that the handheld ultrasound device processes all the obtained ultrasound echo signals into ultrasound data and sends it to the terminal device for display; or the handheld ultrasound device only does partial processing, leaving some of the ultrasound echo signals, and sends both the partially processed ultrasound data and the echo signals to the terminal device, where the terminal device processes them based on the terminal processor to form a final ultrasound image; or, the processor of the handheld ultrasound device performs the first processing of the echo signals to form first ultrasound data, and sends the first ultrasound data to the terminal processor, where the terminal processor performs the second processing of the first ultrasound data to form the final ultrasound image; or, the handheld ultrasound device sends the partially processed ultrasound data and all the echo signals to the terminal device, and the terminal device performs the corresponding processing when necessary. Here, the processor of the terminal device is mainly used to assist the handheld ultrasound device in doing part or all of the processing of the echo signals or images, reducing the load on the processor in the handheld device.

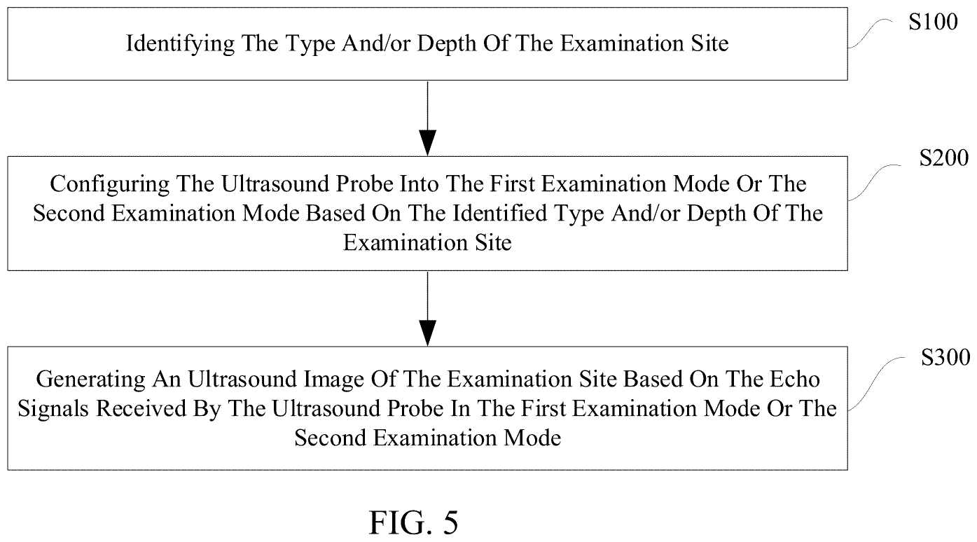

Based on the above-mentioned ultrasound imaging apparatus, an ultrasound imaging method is also provided in some embodiments, as shown in FIG. 5, which includes the following steps:

Step S100: identifying the type and/or depth of the examination site. In the shown embodiment, the depth may be characterized by a distance between the examination site and the body surface, or by a distance between the examination site and the ultrasound probe 110.

Exemplarily, the initial image of the examination site is first obtained; for instance, the ultrasound image of the examination site is generated based on the echo signal returned after the ultrasound probe 110 emits ultrasound waves to the examination site, and this ultrasound image is taken as the initial image of the examination site. Then, based on the initial image of the examination site, the type and/or depth of the examination site is determined.

In some embodiments, based on the obtained initial image of the examination site, the type and/or depth of the examination site are automatically identified according to an image recognition algorithm. Such image recognition algorithms are existing mature technologies and will not be repeated here.

In other embodiments, the display device 118 can provide a display interface for after confirming the type of the image based on experience, based on the user's selection of the type identifier of the examination site displayed in the display interface, the type of the examination site is identified. That is to say, the type of the examination site can be determined through the interaction between the user and the display interface.

In some other embodiments, in the display interface, an input field and/or selection options for depth may also be displayed. Based on the user's interaction with the input field and/or selection options, the depth of the examination site is identified. For example, the input field may be an input box or input window, where the user can enter a value, which is taken as the depth of the examination site. The selection options may be a preset drop-down menu, etc., with preset selectable depths, such as 5 cm, 10 cm, 15 cm, etc. The user may select one of them to input the depth of the examination site.

Step S200: configuring the ultrasound probe into the first examination mode or the second examination mode based on the identified type and/or depth of the examination site.

The following examples illustrate how to determine the examination mode based on the type of the examination site.

When the identified type of examination site is thyroid, carotid artery or blood vessel, the ultrasound probe 110 is set to the first examination mode; and when the identified type of examination site is abdomen or heart, the ultrasound probe 110 is set to the second examination mode. The abdomen mainly refers to some small organs in the abdominal region, such as the intestine, kidney, stomach, and gland. The types of the above-mentioned examination sites are only examples. As can be seen from the above examples, when an examination site belongs to or includes a part that is relatively close to the body surface, the first examination mode is adopted, and when an examination site belongs to or includes a part that is relatively far from the body surface, the second examination mode is adopted.

The following examples illustrate how to determine the examination mode based on depth.

When the identified depth of examination site is within the first depth range, the ultrasound probe 110 is set to the first examination mode; and when the identified depth of examination site is within the second depth range, the ultrasound probe 110 is set to the second examination mode; where the lower limit of the second depth range is greater than the upper limit of the first depth range. For instance, the upper limit of the first depth range is less than or equal to 5 cm, and the lower limit of the second depth range is greater than or equal to 15 cm. Another example is that the upper limit of the first depth range is less than 10 cm, and the lower limit of the second depth range is greater than or equal to 10 cm.

When the ultrasound probe 110 is set to the first examination mode, all the elements in at least three consecutive transducer elements are receiving transducer elements. For example, the ultrasound probe 110 has 128 transducer elements, and all 128 transducer elements are receiving transducer elements each time ultrasound waves are emitted; or, the 128 transducer elements are grouped into four groups of 32 transducer elements each, each time the ultrasound probe 110 emits ultrasound waves, 32 transducer elements in one group function as receiving transducer elements, and the receiving transducer elements in the other three groups function as non-receiving transducer elements, and the four groups take turns to be receiving transducer elements in sequence.

In the first examination mode, due to the continuous receiving transducer elements, the occurrence of grating lobes and/or side lobes in the near field may be unlikely, and thus artifacts may be less likely to appear in the subsequent generated ultrasound images. This mode can provide excellent presentation effects for examination sites that are relatively close to the body surface.

In some embodiments, when the ultrasound probe 110 is set to the second examination mode, the first and the last transducer elements among at least three consecutive transducer elements are both receiving transducer elements, and at least one non-receiving transducer element exists between the first and the last transducer elements. For example, the ultrasound probe 110 has four transducer elements, with the first and the fourth transducer elements being receiving transducer elements and the second and the third transducer elements being non-receiving transducer elements. Another example is that the first, second and fourth transducer elements are receiving transducer elements and the third transducer element is a non-receiving transducer element. In some embodiments, any two receiving transducer elements are not adjacent. For instance, there is one or more non-receiving transducer elements between any two receiving transducer elements. Further, any two receiving transducer elements can be evenly distributed. For example, there is one non-receiving transducer element between every two receiving transducer elements, or there are two non-receiving transducer elements between every two receiving transducer elements.

In some other embodiments, when the ultrasound probe 110 is set to the second examination mode, at least one non-receiving transducer element is present in at least three consecutive elements. In such embodiments, either the first or the last element may not be a receiving transducer element, or neither of them may be a receiving transducer element. For example, the ultrasound probe 110 has 8 transducer elements, with odd-numbered ones being receiving transducer elements and even-numbered ones being non-receiving transducer elements. Similarly, when at least one non-receiving transducer element exists among at least three consecutive elements, in some embodiments, any two receiving transducer elements are not adjacent. For instance, there is one or more non-receiving transducer elements between any two receiving transducer elements. Further, any two receiving transducer elements can also be evenly distributed. For example, there is one non-receiving transducer element between every two receiving transducer elements, or there are two non-receiving transducer elements between every two receiving transducer elements.

It should be noted that when the ultrasound probe 110 is set to the second examination mode, the configuration of which transducer elements are receiving transducer elements and which are non-receiving transducer elements is relative to each reception of the ultrasound waves. For example, when the ultrasound probe 110 has four transducer elements and the ultrasound waves are transmitted twice, during the first reception of the echo signals of the ultrasound waves, the first and fourth transducer elements are receiving transducer elements, while the second and third transducer elements are non-receiving transducer elements, and during the second reception of the echo signals of the ultrasound waves, the first and third transducer elements are receiving transducer elements, and the second and fourth transducer elements are non-receiving transducer elements. In this case, although the third and fourth transducer elements can both be used as receiving transducer elements under the second examination mode, they do not belong to the consecutive two receiving transducer elements under the second examination mode of this embodiment.

Compared with the first examination mode, the second examination mode can increase the receiving aperture under the condition of the same number of receiving transducer elements. For example, with 32 receiving transducer elements, the receiving aperture is 32 transducer elements under the first examination mode. However, under the second examination mode, taking the case of uniformly spacing one non-receiving transducer element as an example, if the odd-numbered transducer elements are set as receiving transducer elements and the even-numbered ones as non-receiving transducer elements, then the 32 receiving transducer elements are the first, third, fifth, . . . , sixty-third transducer elements, while the second, fourth, sixth, . . . , sixty-fourth transducer elements are non-receiving transducer elements. Accordingly, the receiving aperture under the second examination mode is 64 transducer elements. Since the receiving transducer elements are spaced, the receiving interval is doubled, which can improve the penetration effect. That is to say, through this approach, clear images can be obtained in the far field, and good presentation effects can be achieved for examination sites far from the body surface.

From the above description, it can be seen that the ultrasound probe 110 in the above-mentioned embodiments can be used for different image application scenarios: when used for superficial examination, the near-field image is focused on and the first examination mode is adopted; while for applications such as the abdomen where the far-field image is of greater concern, the second examination mode is adopted to achieve an improved penetration effect. Overall, by adopting different examination modes for different application scenarios, the image resolution is enhanced without causing grating lobes and/or side lobes in the near-field area of concern.

In some embodiments, special designs have been made for the connection mode between the physical channels and the transducer elements, thereby achieving the switching between the first examination mode and the second examination mode in some embodiments of the present application. Specifically:

The ultrasound probe 110 may also include multiple physical channels. The number of the multiple transducer elements is M, and the number of the multiple physical channels is N, where N is an even number greater than or equal to 2, and Mis K times N, with K being a positive integer greater than or equal to 2. The ((k−1)*N+n)-th transducer element can be connected to the n-th physical channel, where k=1, 2, . . . , K, and n=1, 2, . . . , N. The physical channels are used to excite the connected transducer elements to emit ultrasound waves or receive the echo signals of ultrasound waves through the connected transducer elements. For example, the value of N can be 8, 16, 32, 64, 128, 192 or 256; the value of M can be 64, 80, 96, 128, 192, 256 or 336, etc.

For example, with N being 64, K being 2, and M being 256, FIG. 4 shows the connection mode between 64 physical channels and the first 128 transducer elements, while the connection manner of the 64 physical channels to the latter 128 transducer elements is similar. Specifically: the first physical channel is connected to the first and 65th transducer elements, the second physical channel is connected to the second and 66th transducer elements, . . . , the 32nd physical channel is connected to the 32nd and 96th transducer elements, the 35th physical channel is connected to the 35th and 99th transducer elements, . . . , and the 64th physical channel is connected to the 64th and 128th transducer elements. In the following examples, the connection mode between physical channels and transducer elements as shown in FIG. 4 is taken as an example. In FIG. 4, the receive selection switch on the first physical channel is closed, the receive selection switch on the second physical channel is open, and so on. The receive selection switch on the first physical channel is represented by TX1, the receive selection switch on the second physical channel is represented by TX2, and so on. RX represents the transmit selection switch, for example, RX1 represents the first transmit selection switch.

When the ultrasound probe 110 is set to the first examination mode, the first to the N-th, the (N+1)-th to the (2*N)-th, . . . , or the ((K−1)*N+1)-th to the (K*N)-th transducer elements are all receiving transducer elements.

For example, under the first examination mode, the receive selection switches on the first physical channel to the 64th physical channel are controlled to be closed, and then the first physical channel is mapped to the first element, the second physical channel is mapped to the second element, and so on, so that the first element to the 64th element are receiving transducer elements. The above-mentioned “map” can be achieved through software in this field and will not be repeated here. It can be understood that the first physical channel can also be mapped to the 65th transducer element, the second physical channel to the 66th transducer element, and so on, so that the 65th transducer element to the 128th transducer element are receiving transducer elements. It can be seen that the receiving aperture formed above includes 64 consecutive transducer elements.

When the ultrasound probe 110 is set to the second examination mode, the 1st, 3rd, . . . , to (N−1)-th and the (N+2)-th, (N+4)-th, . . . , to (2*N)-th transducer elements are all receiving transducer elements, while the 2nd, 4th, . . . , to N-th and the (N+1)-th, (N+3)-th, . . . , to (2*N−1)-th transducer elements are all non-receiving transducer elements; or, the (N+1)-th, (N+3)-th, . . . , to (2*N−1)-th and the (2*N+2)-th, (2*N+4)-th, . . . , to (3*N)-th transducer elements are all receiving transducer elements, while the (N+2)th, (N+4)th, . . . , to (2*N)th and the (2*N+1)th, (2*N+3)th, . . . , (3*N−1)th transducer elements are all non-receiving transducer elements; . . . , or, the ((K−2)*N+1)-th, ((K−2)*N+3)-th, . . . , to ((K−1)*N−1)-th and the ((K−1)*N+2)-th, ((K−1)*N+4)-th, . . . to (K*N)-th transducer elements are all receiving transducer elements, while the ((K−2)*N+2)-th, ((K−2)*N+4)-th, . . . to ((K−1)*N)-th and the ((K−1)*N+1)-th, ((K−1)*N+3)-th, . . . to (K*N−1)-th transducer elements are all non-receiving transducer elements.

Continuing with the connection method in FIG. 4 as an example, under the second examination mode, the receive selection switches on the first physical channel to the 64th physical channel are closed; then, the first physical channel is controlled to map to the first transducer element, the second physical channel to map to the 66th transducer element, the third physical channel to map to the 3rd transducer element, the fourth physical channel to map to the 68th transducer element, . . . , the 35th physical channel to map to the 35th transducer element, . . . , and the 64th physical channel to map to the 128th transducer element. It can be seen that the receiving aperture formed in this way is 128 transducer elements, with the receiving transducer elements distributed at intervals. This special receiving method can double the receiving aperture and improve the imaging lateral resolution.

Step S300: generating an ultrasound image of the examination site based on the echo signals received by the ultrasound probe under the first examination mode or the second examination mode.

In some other embodiments, the echo signals received by the ultrasound probe 110 in the first or second examination mode and/or the ultrasound data generated based on the echo signals are sent to a terminal device that is in communication with the ultrasound imaging apparatus. The terminal device includes, but is not limited to, a smart phone or a tablet computer. The terminal device generates an ultrasound image of the examination site based on the received echo signals and/or ultrasound data. For example, the terminal device includes a terminal processor, which generates an ultrasound image of the examination site based on the received echo signals and/or ultrasound data. The ultrasound image generated by the terminal device can be sent back to the ultrasonic imaging device for display, or the terminal device can provide its own display interface for showing the ultrasound image.

In some embodiments, in response to a user's interactive operation on the display interface, the ultrasound probe 110 enters the first examination mode or the second examination mode. For example, the display interface shows the identifier corresponding to the first examination mode or the identifier corresponding to the second examination mode, the examination mode corresponding to the identifier may be entered after it is selected by the user.

In some embodiments, it is also possible not to perform recognition on the initial image first, for instance, when the user clearly knows the part they want to examine. The user can directly manually select the corresponding examination mode identifier on the display interface or perform an operation such as depth input field/selection options to enter into the corresponding examination mode, which is selected based on the examination mode identifier or the depth.

The present disclosure refers to various exemplary embodiments for illustrative purposes. However, those skilled in the art will recognize that modifications and alterations may be made to these embodiments without departing from the scope of the disclosure. For instance, individual operational steps and components for performing such steps may be implemented in diverse manners depending on specific applications or considerations of cost functions associated with system operations (e.g., one or more steps may be deleted, modified, or consolidated with other steps).

Moreover, as understood by those skilled in the art, the principles disclosed may be embodied in a computer program product stored on a non-transitory computer-readable storage medium preloaded with computer-readable program code. Any tangible, non-transitory computer-readable storage medium may be utilized, including but not limited to: magnetic storage devices (e.g., hard disks, floppy disks); optical storage devices (e.g., CD-ROMs, DVDs, Blu-ray discs); and flash memory devices. The computer program instructions may be loaded onto a general-purpose computer, special-purpose computer, or other programmable data processing apparatus to create a machine, such that the instructions executed on the computer or programmable apparatus produce means for implementing specified functions. These instructions may also reside in a computer-readable memory, directing the computer or programmable apparatus to operate in a defined manner, thereby forming an article of manufacture comprising functional implementation means. Furthermore, the computer program instructions may be executed on a computer or programmable data processing apparatus to generate a computer-implemented process, wherein the executed instructions provide steps for realizing the specified functionality, including but not limited to: technical improvements in data processing efficiency (e.g., optimized memory allocation); and enhanced accuracy in algorithmic execution (e.g., reduced error margins in machine learning models).

While the principles disclosed herein have been illustrated through various embodiments, it should be understood that structural configurations, material selections, and component proportions particularly suited to specific operational environments may be modified without departing from the scope and spirit of the disclosure. Such modifications, along with other adaptations or adjustments, shall be encompassed within the scope of the present disclosure.

The foregoing detailed description has been described with reference to various embodiments. However, those skilled in the art will recognize that modifications and variations may be made without departing from the scope of the disclosure. Accordingly, the description of the disclosure shall be interpreted in an illustrative rather than restrictive sense, and all such modifications are intended to be included within its scope. Similarly, discussions of advantages, alternative solutions to problems, and operational benefits associated with the embodiments are provided above. Nevertheless, benefits, advantages, solutions to problems, and any elements that may produce such effects or render them more explicit shall not be construed as critical, required, or essential. Furthermore, the term ‘coupled’ and its derivatives encompass physical connections (e.g., mechanical joints), electrical connections (e.g., circuit interconnects), magnetic linkages (e.g., inductive coupling), optical interfaces (e.g., fiber-optic alignment), communication channels (e.g., wireless protocols), functional integrations (e.g., software APIs), and any other form of association that achieves operational interaction.

Those skilled in the art will recognize that numerous modifications to the details of the above-described embodiments may be made without departing from the fundamental principles of the disclosed subject matter. Accordingly, the scope of the present disclosure shall be determined solely by the claims and their legal equivalents.

What is claimed is:

1. An ultrasound imaging apparatus, comprising:
an ultrasound probe comprising a plurality of transducer elements, wherein the ultrasound probe is at least configured to emit ultrasound waves to an examination site of an object under examination through at least one of the plurality of transducer elements; and
a processor, configured for:
identifying a type and/or depth of the examination site;
configuring the ultrasound probe into a first examination mode or a second examination mode based on the identified type and/or depth of the examination site, wherein: at least three consecutive transducer elements are present in the plurality of transducer elements: when the ultrasound probe is configured into the first examination mode, all the at least three consecutive transducer elements are activated as receiving transducer elements; when the ultrasound probe is configured into the second examination mode, a first transducer element and a last transducer element within the at least three consecutive transducer elements are activated as receiving transducer elements, and at least one non-receiving transducer element is present between the first transducer element and the last transducer element; wherein the receiving transducer elements are configured to receive echo signals of the ultrasound waves returned from the examination site, and the non-receiving transducer element is configured not to receive the echo signals of the ultrasound waves returned from the examination site; and
generating an ultrasound image of the examination site based on the echo signals received by the ultrasound probe under the first examination mode or the second examination mode; and/or, sending the echo signals received by the ultrasound probe under the first examination mode or the second examination mode and/or sending ultrasound data from the echo signals received by the ultrasound probe in the first examination mode or the second examination mode to a terminal device communicatively connected to the ultrasound imaging apparatus, the terminal device generating an ultrasound image of the examination site based on the received echo signals and/or the received ultrasound data.

2. The ultrasound imaging apparatus according to claim 1, wherein configuring the ultrasound probe into the first examination mode or the second examination mode based on the identified type of the examination site, comprises:
configuring the ultrasound probe into the first examination mode when the identified type of the examination site comprises a thyroid, carotid artery, or a blood vessel; and
configuring the ultrasound probe into the second examination mode when the identified type of the examination site comprises an abdomen or a heart.

3. The ultrasound imaging apparatus according to claim 1, wherein configuring the ultrasound probe into the first examination mode or the second examination mode based on the identified depth of the examination site, comprises:
configuring the ultrasound probe into the first examination mode when the identified depth of the examination site is within a first depth range;
configuring the ultrasound probe into the second examination mode when the identified depth of the examination site is within a second depth range;
wherein a lower limit of the second depth range is greater than an upper limit of the first depth range.

4. The ultrasound imaging apparatus according to claim 3, wherein the upper limit of the first depth range is less than or equal to 5 cm, and the lower limit of the second depth range is greater than or equal to 15 cm.

5. The ultrasound imaging apparatus according to claim 1, wherein when the ultrasound probe is configured into the second examination mode and at least two non-receiving transducer elements are present between the first transducer element and the last transducer element, any two of the receiving transducer elements are non-adjacent.

6. The ultrasound imaging apparatus according to claim 1, wherein when the ultrasound probe is configured into the second examination mode and at least three non-receiving transducer elements are present between the first transducer element and the last transducer element, the receiving transducer elements are uniformly spaced.

7. The ultrasound imaging apparatus according to claim 1, wherein the ultrasound probe further comprises a plurality of physical channels; a number of the plurality of transducer elements is M, a number of the plurality of physical channels is N, where N is an even integer greater than or equal to 2, Mis K times of N, and K is a positive integer greater than or equal to 2; ((k−1)*N+n)-th transducer element is operably connected to n-th physical channel, where k=1, 2, . . . , K, and n=1, 2, . . . , N; each physical channel is configured to excite its connected transducer element(s) to emit ultrasound waves, or to receive echo signals of ultrasound waves through its connected transducer element(s);
when the ultrasound probe is configured into the first examination mode, first to N-th, (N+1)-th to (2*N)-th, . . . , or ((K−1)*N+1)-th to (K*N)-th transducer elements are all receiving transducer elements;
when the ultrasound probe is configured into the second examination mode: first, third, . . . , (N−1)-th and (N+2)-th, (N+4)-th, . . . , (2*N)-th transducer elements are receiving transducer elements, and second, fourth, . . . , N-th and (N+1)-th, (N+3)-th, . . . , (2*N−1)-th transducer elements are non-receiving transducer elements;
or, (N+1)-th, (N+3)-th, . . . , (2*N−1)-th, and (2*N+2)-th, (2*N+4)-th, . . . , (3*N)-th transducer elements are receiving transducer elements, and (N+2)-th, (N+4)-th, . . . , (2*N)-th and (2*N+1)-th, (2*N+3)-th, . . . , (3*N−1)-th transducer elements are non-receiving transducer elements;
. . . ,
or, the ((K−2)*N+1)-th, ((K−2)*N+3)-th, . . . , ((K−1)*N−1)-th and ((K−1)*N+2)-th, ((K−1)*N+4)-th, . . . , (K*N)-th transducer elements are receiving transducer elements, and the ((K−2)*N+2)-th, ((K−2)*+4)-th, . . . , ((K−1)*N)-th and ((K−1)*N+1)-th, ((K−1)*N+3)-th, . . . , (K*N−1)-th are non-receiving transducer elements.

8. The ultrasound imaging apparatus according to claim 7, wherein a value of N is 8, 16, 32, 64, 128, 192, or 256; or a value of M is 64, 80, 96, 128, 192, 256, or 336.

9. The ultrasound imaging apparatus according to claim 1, wherein before identifying the type and/or depth of the examination site, the processor is further configured for: acquiring an initial image of the examination site;
identifying the type and/or depth of the examination site comprises:
automatically identifying the type and/or depth of the examination site based on the acquired initial image of the examination site using an image recognition algorithm; or,
identifying, based on the acquired initial image of the examination site, the type and/or depth of the examination site in response to an interactive operation by a user on a display interface of the ultrasound imaging apparatus.

10. The ultrasound imaging apparatus according to claim 9, wherein identifying, based on the acquired initial image of the examination site, the type of the examination site in response to an interactive operation by a user on a display interface of the ultrasound imaging apparatus, comprises:
displaying a type identifier of the examination site on the display interface; and
identifying the type of the examination site based on a user selection of the type identifier.

11. The ultrasound imaging apparatus according to claim 9, wherein identifying, based on the acquired initial image of the examination site, the depth of the examination site in response to an interactive operation by a user on a display interface of the ultrasound imaging apparatus, comprises:
displaying a depth input field and/or depth selection options on the display interface; and
identifying the depth of the examination site based on the interactive operation by the user on the depth input field and/or the depth selection options.

12. The ultrasound imaging apparatus according to claim 1, wherein the ultrasound imaging apparatus is a handheld ultrasound imaging device, the handheld ultrasound imaging device is communicatively connected to the terminal device, and the processor is integrated in the ultrasound probe.

13. The ultrasound imaging apparatus according to claim 12, wherein the terminal device includes a terminal processor, the terminal processor is configured to generate the ultrasound image of the examination site based on the received echo signals and/or the received ultrasound data.

14. The ultrasound imaging apparatus according to claim 12, wherein the terminal device is configured to provide a display interface configured to display the ultrasound image.

15. An ultrasound imaging apparatus, comprising:
an ultrasound probe comprising a plurality of transducer elements, wherein the ultrasound probe is at least configured to emit ultrasound waves to an examination site of an object under examination by at least one of the plurality of transducer elements; the ultrasound probe at least comprises a first examination mode and a second examination mode; at least three consecutive transducer elements are present in the plurality of transducer elements: when the ultrasound probe is configured into the first examination mode, all the at least three consecutive transducer elements are activated as receiving transducer elements; when the ultrasound probe is configured into the second examination mode, a first transducer element and a last transducer element within the at least three consecutive transducer elements are activated as receiving transducer elements, and at least one non-receiving transducer element is present between the first transducer element and the last transducer element; wherein the receiving transducer elements are configured to receive echo signals of the ultrasound waves returned from the examination site, and the non-receiving transducer element(s) is (are) configured not to receive the echo signals of the ultrasound waves returned from the examination site; and
a processor configured to cause the ultrasound probe to enter into a predetermined examination mode in response to an interactive operation by a user on a display interface, comprising: based on different interactive operations, respectively:
enabling the ultrasound probe to enter into the first examination mode, so as to perform imaging on the examination site under the first examination mode;
enabling the ultrasound probe to enter into the second examination mode, so as to perform imaging on the examination site under the second examination mode; and
wherein the display interface is provided by a terminal device communicatively connected to the ultrasound probe or provided by the ultrasound apparatus.

16. The ultrasound imaging apparatus according to claim 15, wherein the display interface is configured to display at least one first examination mode identifier and at least one second examination mode identifier, wherein the first examination mode identifier corresponds to the first examination mode of the ultrasound probe, and the second examination mode identifier corresponds to the second examination mode of the ultrasound probe;
the processor is configured to enable the ultrasound probe to enter into the first examination mode in response to the interactive operation by the user on the at least one first examination mode identifier on the display interface; or, the processor is configured to enable the ultrasound probe to enter into the second examination mode in response to the interactive operation by the user on the at least one second examination mode identifier on the display interface.

17. The ultrasound imaging apparatus according to claim 16, wherein the first examination mode identifier comprises a thyroid examination mode identifier, a carotid artery examination mode identifier, or a blood vessel examination mode identifier; and the second examination mode comprises an abdominal examination mode identifier, or a cardiac examination mode identifier.

18. The ultrasound imaging apparatus according to claim 15, wherein
the display interface is configured to display a depth input field and/or depth selection options; and
the processor is configured to enable the ultrasound probe to enter into the first examination mode or the second examination mode in response to the interactive operation by the user on the depth input field and/or the depth selection options.

19. The ultrasound imaging apparatus according to claim 15, wherein the ultrasound probe further comprises a plurality of physical channels; a number of the plurality of transducer elements is M, a number of the plurality of physical channels is N, where N is an even integer greater than or equal to 2, Mis K times of N, and K is a positive integer greater than or equal to 2; ((k−1)*N+n)-th transducer element is operably connected to n-th physical channel, where k=1, 2, . . . , K, and n=1, 2, . . . , N; each physical channel is configured to excite its connected transducer element(s) to emit ultrasound waves, or receive echo signals of ultrasound waves through its connected transducer element(s);
when the ultrasound probe is configured into the first examination mode, first to N-th, (N+1)-th to (2*N)-th, . . . , or ((K−1)*N+1)-th to (K*N)-th transducer elements are all receiving transducer elements;
when the ultrasound probe is configured into the second examination mode: first, third, . . . , (N−1)-th and (N+2)-th, (N+4)-th, . . . , (2*N)-th transducer elements are receiving transducer elements, and second, fourth, . . . , N-th and (N+1)-th, (N+3)-th, . . . , (2*N−1)-th transducer elements are non-receiving transducer elements;
or, (N+1)-th, (N+3)-th, . . . , (2*N−1)-th, and (2*N+2)-th, (2*N+4)-th, . . . , (3*N)-th transducer elements are receiving transducer elements, and (N+2)-th, (N+4)-th, . . . , (2*N)-th and (2*N+1)-th, (2*N+3)-th, . . . , (3*N−1)-th transducer elements are non-receiving transducer elements;
. . . ,
or, ((K−2)*N+1)-th, ((K−2)*N+3)-th, . . . , ((K−1)*N−1)-th and ((K−1)*N+2)-th, ((K−1)*N+4)-th, . . . , (K*N)-th transducer elements are receiving transducer elements, and ((K−2)*N+2)-th, ((K−2)*+4)-th, . . . , ((K−1)*N)-th and ((K−1)*N+1)-th, ((K−1)*N+3)-th, . . . , (K*N−1)-th are non-receiving transducer elements.

20. An ultrasound imaging method, comprising:
identifying a type and/or depth of an examination site of an object under examination;
configuring an ultrasound probe into a first examination mode or a second examination mode based on the identified type and/or depth of the examination site, wherein the ultrasound probe comprises a plurality of transducer elements; the ultrasound probe is at least configured to emit ultrasound waves to the examination site through at least one of the plurality of transducer elements; at least three consecutive transducer elements are present in the plurality of transducer elements: when the ultrasound probe is configured into the first examination mode, all the at least three consecutive transducer elements are activated as receiving transducer elements; when the ultrasound probe is configured into the second examination mode, a first transducer element and a last transducer element within the at least three consecutive transducer elements are activated as receiving transducer elements, and at least one non-receiving transducer element is present between the first transducer element and the last transducer element; wherein the receiving transducer element is configured to receive echo signals of the ultrasound waves returned from the examination site, and the non-receiving transducer element(s) is (are) configured not to receive echo signals of the ultrasound waves returned from the examination site; and
generating an ultrasound image of the examination site based on the echo signals received by the ultrasound probe under the first examination mode or the second examination mode; and/or, sending the echo signals received by the ultrasound probe under the first examination mode or the second examination mode and/or sending ultrasound data from the echo signals received by the ultrasound probe under the first examination mode or the second examination mode to a terminal device communicatively connected to the ultrasound imaging apparatus, wherein the terminal device generates an ultrasound image of the examination site based on the received echo signals and/or the received ultrasound data.
